(19) United States
(12) Patent Application Publication
(10) Pub. No.: US 2020/0060648 A1
(43) Pub. Date: Feb. 27, 2020

(54) SYSTEM FOR PULSE WAVE MEASUREMNT AND ALIGNMENT GUIDANCE METHOD THEREOF

(71) Applicant: NATIONAL CHENG KUNG UNIVERSITY, Tainan (TW)

(72) Inventors: CHIH-CHUNG HUANG, Tainan (TW); PO-YANG LEE, Tainan (TW); YI-HSIANG CHUANG, Kaohsiung (TW)

(21) Appl. No.: 16/106,016

(22) Filed: Aug. 21, 2018

(51) Int. Cl. A61B 8/00; A61B 8/02; A61B 8/08; A61B 8/06

(52) U.S. Cl. CPC A61B 8/4254; A61B 8/54; A61B 8/02; A61B 8/06; A61B 8/4488; A61B 8/488; A61B 8/461

(57) ABSTRACT

An ultrasound transducer system includes an alignment-guidance transducer device and a control device. The alignment-guidance transducer device includes a transducer module for emitting ultrasound waves and receiving ultrasound signals reflected from a target hollow organ, and an indicator module for indicating a state of alignment between the alignment-guidance transducer device and the target hollow organ. The control device is in communication with the alignment-guidance transducer device for processing the ultrasound signals received from the transducer module and transmitting instructions to the indicator module according to the processed ultrasound signals, so as to guide a user to align the ultrasound transducer device to an extension direction of the target hollow organ. Processing of the ultrasound signals includes analyzing the ultrasound signals to obtain characteristics of the target hollow organ, and determining the levels of alignment of the transducer module to the hollow target organ according to the characteristics.

### FIELD OF THE DISCLOSURE

The present disclosure relates to a system for pulse wave measurement and alignment guidance method thereof, and more particularly to a pulse wave measurement system having an ultrasound transducer device that provides alignment guidance.

### BACKGROUND OF THE DISCLOSURE

Vascular pulse wave has long been considered a useful reference for cardiovascular assessment. For example, arterial pulse wave analysis allows non-invasive measurement of pulse wave velocity, estimation of artery elasticity, and assessment of the risk of complications or diseases manifested with abnormal blood pulse wave. Pulse wave analysis can also be utilized for functional assessment of other hollow organs, for example, ureter.

Conventionally, vascular pulse wave may be detected by pressure cuffs, tonometry, high-resolution Doppler ultrasound devices or photoplethysmography. However, a list of shortcomings still hamper the use of pulse wave analysis on clinical applications. For example, while pressure cuff has been most commonly applied around brachial artery for systolic and diastolic blood pressure, waveform detected by a pressure cuff could be delayed and distorted by subcutaneous tissue and the cuff itself. Similarly, tonometry improves response latency, but encounters waveform distortion problems as well. Photoplethysmography detects pulse wave by blood volume change, but hardly distinguishes vessel wall characteristics and flow velocity.

As compared with pressure cuffs, tonometry and photoplethysmograph, high resolution ultrasound devices allow a more comprehensive cardiovascular assessment with structural and hemodynamic measurements. However, ultrasound devices require professionals undergoing an extremely shallow learning curve and mastery of knobology. For example, pulse wave detection requires strict alignment of the ultrasound probe to the blood vessel and complicated tuning of imaging parameters, which are difficult for the users to accomplish accurate measurement and standardized procedure. Doppler ultrasound devices, such as those provided by Hoctor et al. in U.S. Pat. No. 7,621,876, Benthin et al. in U.S. Pat. No. 4,660,564, Elliot et al. in U.S. Patent Publication No. 2015/0374249 and Wilmering in U.S. Patent Publication No. 2014/0187992, have mentioned the importance of alignment between the ultrasound probe and the vessel. Unfortunately, those literatures fail to disclose how alignment can be achieved.

To facilitate the alignment process, Rinderknecht et al. in U.S. Patent Publication No. 2016/0135697 affixes or implants a marker at an optimal spot for signal acquisition. Yang in U.S. Patent Publication No. 2014/0276123 adopts an array of pulse wave sensors to increase coverage of an intended arterial site. However, as additional components or processes are required, neither of these improvements has provided a cost and time effective solution.

Alternatively, Kantorovich et al. in U.S. Pat. No. 6,261,233 reduces the burden of strict alignment by processing the acquired Doppler-shifted reflection signals through a series of mathematical calculations. However, Kantorovich still requires the ultrasound probe to be longitudinally aligned with the major axis of the target vessel; yet, there is no way for the user to confirm the status of the alignment.

### BRIEF SUMMARY OF THE DISCLOSURE

An objective of some embodiments of the present disclosure is to provide a cost-effective and intuitive system for precise measurement of vascular pulse waves.

Another objective of some embodiments of the present disclosure is to provide an alignment guidance method for the system for precise measurement of vascular pulse waves.

An embodiment of the present disclosure provides an ultrasound transducer device having alignment guidance function. The ultrasound transducer device includes a transducer module for emitting ultrasound waves and receiving ultrasound signals reflected from a target hollow organ, and an indicator module for indicating a state of alignment between the ultrasound transducer device and the target hollow organ. The ultrasound transducer device is so configured that the ultrasound transducer device is communicable with a control device that is capable of processing the ultrasound signals received from the transducer module and transmitting instructions to the indicator module according to the processed ultrasound signals.

Another embodiment of the present disclosure provides an ultrasound transducer system. The system includes an alignment-guidance transducer device and a control device. The alignment-guidance transducer device includes a transducer module for emitting ultrasound waves and receiving ultrasound signals reflected from a target hollow organ, and an indicator module for indicating a state of alignment between the alignment-guidance transducer device and the target hollow organ. The control device is in communication with the alignment-guidance transducer device for processing the ultrasound signals received from the transducer module and transmitting instructions to the indicator module according to the processed ultrasound signals.

Preferably, the transducer module includes a plurality of ultrasound elements for emitting the ultrasound waves and receiving the ultrasound signals.

Preferably, each of the ultrasound elements is an actuator element, a sensor element or a transducer element.

Preferably, the ultrasound elements emit the ultrasound waves in a synchronized or phase-delayed manner.

Preferably, the ultrasound waves emitted by at least a portion of the ultrasound elements are continuous waves or pulse waves.

Preferably, the transducer module is configured for detecting one or more characteristics of the target hollow organ. The characteristics include wall of the target hollow organ and displacement of the wall.

Preferably, the transducer module is further configured for measuring one or more characteristics of the target hollow organ. The characteristics include pulse wave velocity.

Preferably, the ultrasound elements are arranged in a substantially linear pattern.

Preferably, the ultrasound elements are orthogonally arranged.

Preferably, the indicator module of the ultrasound transducer device includes a plurality of output units. Each of the output units is a visual output unit or an audio output unit.

Preferably, the state of alignment is indicated by stationary signals or dynamic signals.

An embodiment of the present disclosure provides an alignment guidance method implemented by the ultrasound transducer system. The method includes the steps of: (S1) emitting ultrasound waves to a target hollow organ by the transducer module of the alignment-guidance transducer device; (S2) receiving ultrasound signals reflected from the target hollow organ by the transducer module; (S3) processing the ultrasound signals by the control device to obtain levels of alignment of the transducer module to the target hollow organ; and (S4) indicating a state of alignment of the alignment-guidance transducer device according to the levels of alignment of the transducer module by the indicator module of the alignment-guidance transducer device.

Preferably, the step S3 includes the steps of: (S31) analyzing the ultrasound signals to obtain one or more characteristics of the target hollow organ; and (S32) determining the levels of alignment of the transducer module to the target hollow organ according to the characteristics. The characteristics include wall of the target hollow organ and displacement of the wall.

Preferably, the levels of alignment of the ultrasound elements to the target hollow organ are defined by distances between each of the ultrasound elements and the target hollow organ, the angle formed by the ultrasound elements relative to the extension direction of the target hollow organ, or the signal-to-noise ratio of the received ultrasound signals.

Preferably, the step S3 further includes a step of: (S33) processing the ultrasound signals to obtain measurement of one or more characteristics of the target hollow organ. The characteristics include pulse wave velocity.

In sum, the ultrasound transducer system according to various embodiments of the present invention utilizes an ultrasound transducer device having an intuitive alignment guidance function to facilitate alignment of the transducer device to the target organ and ensure accuracy of pulse wave measurements by the ultrasound transducer system.

### BRIEF DESCRIPTION OF THE DRAWINGS

The accompanying drawings illustrate one or more embodiments of the present invention and, together with the written description, explain the principles of the present invention. Wherever possible, the same reference numbers are used throughout the drawings to refer to the same or like elements of an embodiment.

FIG. 1 is a schematic illustration of an ultrasound transducer system in accordance with an embodiment of the present disclosure;

FIG. 2 is a schematic illustration of an alignment-guidance transducer device of the ultrasound transducer system in accordance with an embodiment of the present disclosure;

FIG. 3 is a schematic illustration of a transducer module of the alignment-guidance transducer device in accordance with an embodiment of the present disclosure;

FIGS. 4A and 4B are schematic illustrations of the functions of ultrasound elements in the transducer module of the alignment-guidance transducer device in accordance with some embodiments of the present disclosure;

FIG. 4C is a schematic illustration of the arrangement of the ultrasound elements in the alignment-guidance transducer device in accordance with an embodiment of the present disclosure;

FIG. 5 is a schematic illustration of the configuration of a control device of the ultrasound transducer system in accordance with an embodiment of the present disclosure;

FIGS. 6A and 6B are schematic illustrations depicting the operation of an indicator module of the alignment-guidance transducer device in accordance with some embodiments of the present disclosure;

FIG. 7A is a schematic illustration depicting the operation of the alignment-guidance transducer device of the ultrasound transducer system in accordance with an embodiment of the present disclosure;

FIG. 7B is a schematic illustration depicting an ultrasound signal acquired by the alignment-guidance transducer device of the ultrasound transducer system in accordance with an embodiment of the present disclosure; and

FIG. 7C is a schematic illustration depicting a displacement waveform acquired by the alignment-guidance transducer device of the ultrasound transducer system in accordance with an embodiment of the present disclosure.

In accordance with common practice, the various described features are not drawn to scale and are drawn to emphasize features relevant to the present disclosure. Like reference characters denote like elements throughout the figures and text.

### DETAILED DESCRIPTION OF THE PREFERRED EMBODIMENTS

The present invention will now be described more fully hereinafter with reference to the accompanying drawings illustrating various exemplary embodiments of the invention. The present invention may, however, be embodied in many different forms and should not be construed as limited to the embodiments set forth herein. Rather, these embodiments are provided so that this disclosure will be thorough and complete, and will fully convey the scope of the disclosure to those skilled in the art. Like reference numerals refer to like elements throughout.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the terms “and/or” and “at least one” include any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The term “ultrasound” mentioned in the present disclosure is defined as use of sound wave that is produced by a piezoelectric transducer encased in a probe to evaluate the circulatory system of the body. Specifically, vascular ultrasound is a noninvasive method to identify blockages in arteries and veins and detect blood clots. The term “alignment” mentioned in the present disclosure refers to the state of adjustment of the position of the ultrasound transducer device in relation to the target hollow organ.

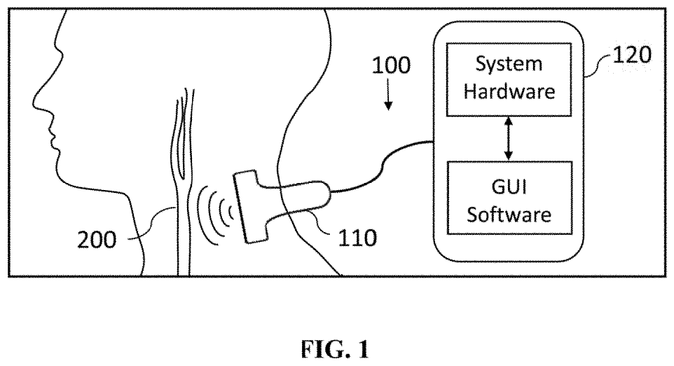

Referring to FIG. 1. An embodiment of the present disclosure provides an ultrasound transducer system 100 for detecting and measuring the characteristics of a target hollow organ 200 or the fluids within the target hollow organ 200. The ultrasound transducer system 100 includes an alignment-guidance transducer device 110 and a control device 120 in communication with the alignment-guidance transducer device 110. The alignment-guidance transducer device 110 is configured to detect the characteristics of the target hollow organ 200 and to guide the user to align the alignment-guidance transducer device 110 to an extension direction of the target hollow organ 200. In a preferred embodiment, the alignment-guidance transducer device 110 is further configured to measure one or more other characteristics of the target hollow organ 200.

In the embodiment, the target hollow organ 200 may be a tube or a canal structure in the body of a mammalian subject. More specifically, the target hollow organ 200 may have biological fluid circulating or flowing within the lumen of the target hollow organ 200. For example, the target hollow organ 200 may be blood vessels or urinary tracts. The blood vessels may include aorta, arteries, arterioles, venules, veins and vena cavae. Specifically, the artery may be, but not limited to, pulmonary artery, carotid artery, subclavian artery, vertebral artery, axillary artery, brachial artery, radial artery, ulnar artery, iliac artery, femoral artery, popliteal artery, tibial artery, arteria dorsalis pedis, renal artery or coronary artery. The urinary tract may be ureter or urethra. Furthermore, the hollow organ 200 may be a part of a fetus within a maternal body.

The characteristics of the target hollow organ 200 may be acoustical characteristics, mechanical characteristics, or fluid dynamic characteristics. For example, acoustical characteristics may be acoustical absorption, echogenicity, acoustical attenuation, acoustical impedance or speed of sound. Furthermore, the acoustical characteristics may be measured along with a predetermined frequency or a series of varying frequencies. Mechanical characteristics may be, for example, thickness, elasticity or stiffness. Fluid dynamic characteristics may be flow velocity or flow waveform. It is contemplated that the characteristics of the target hollow organ may be measured in a time dependent fashion or change in unit time. For example, tracking the displacement of the wall of the target hollow organ having specific acoustical impedance over time can be recorded as pulse waveform.

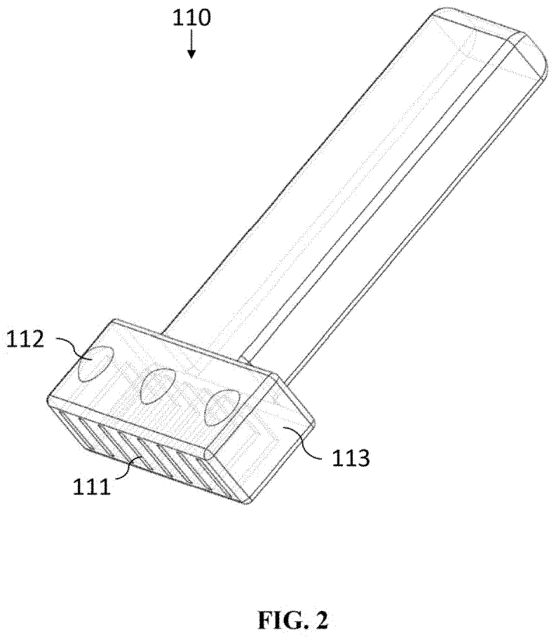

Referring to FIG. 2. In the embodiment, the alignment-guidance transducer device 110 includes a transducer module 111, an indicator module 112, and a housing 113. The indicator module 112 is disposed on a side of the housing 113. The transducer module 111 is configured to emit ultrasound waves and receive ultrasound signals reflected from the target hollow organ 200. The indicator module 112 is configured to indicate a state of alignment between the alignment-guidance transducer device 110 and the target hollow organ 200. The housing 113 accommodates the transducer module 111, the indicator module 112 and provides a handle for the user to hold onto when operating the alignment-guidance transducer device 110. The housing 113 may have a curved surface to conform to the contour of the skin of the subject close to the target hollow organ.

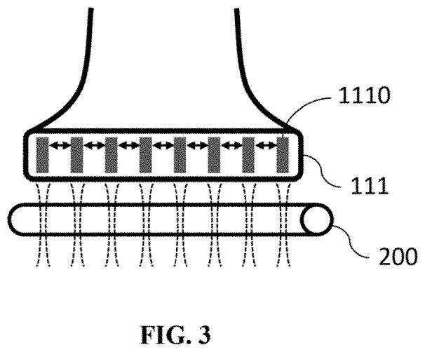

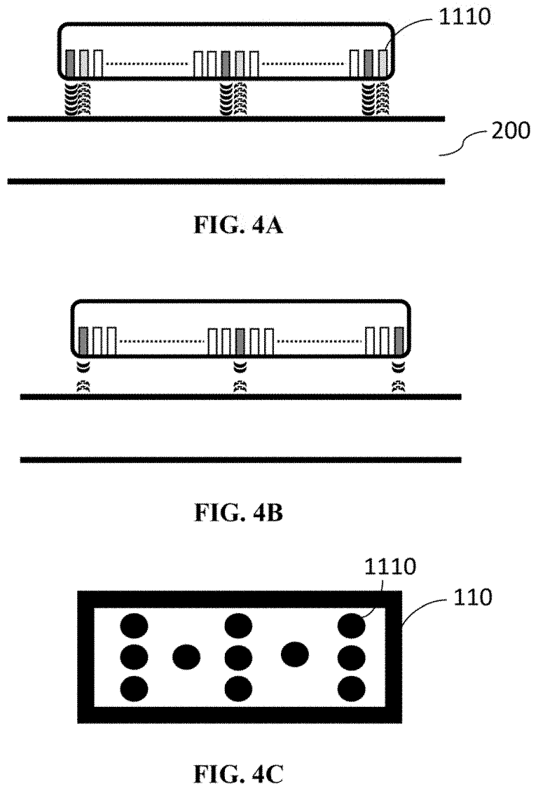

Referring to FIG. 3 and FIG. 4. As illustrated in FIG. 3, the transducer module 111 includes a plurality of ultrasound elements 1110, each of which may be an actuator element for emitting ultrasound waves, a sensor element for acquiring ultrasound signals or a transducer element for emitting ultrasound waves and acquiring ultrasound signals. The ultrasound elements may be configured for emitting ultrasound waves and/or receiving ultrasound signals reflected from the target hollow organ 200 individually or in a synchronized or phase-delayed manner. For example, as illustrated in FIG. 4A, some of the ultrasound elements may be configured for ultrasound waves emission while some others are configured for signal acquisition; as illustrated in FIG. 4B, the ultrasound elements may also be configured for both ultrasound wave emission and signal acquisition. The ultrasound elements 1110 may be piezoelectric micromachined ultrasonic transducers (PMUT), such as polyvinylidene fluoride (PVDF) transducer or lead zirconate titanate (PZT) transducer, or capacitive micromachined ultrasonic transducers (CMUT). The ultrasound elements 1110 may be fabricated in an array (e.g., by a CMOS process) and be physically or electrically grouped into several transducer units.

The ultrasound waves emitted by the ultrasound elements 1110 may be continuous waves or pulse waves, and properties thereof may be optimized for detecting one or more characteristics of the target hollow organ 200. For example, when one or more of the ultrasound elements 1110 are configured to emit continuous waves, the continuous waves may be unmodulated waves with constant frequency and amplitude, frequency-modulated waves, amplitude-modulated waves or phase-modulated waves. Alternatively, the continuous waves may be scanning waves with varying frequencies, amplitudes or phases for acquiring a power spectrum. When one or more of the ultrasound elements 1110 are configured to emit pulse waves, the pulse waves may be of various pulse repetition periods, burst cycles, and burst waveforms for measurement of flow velocity, wall displacement, or backscattering signals from vessel walls or fluid in a specific location of a lumen. The reflected pulse wave signals may further be gated by a depth selector.

Referring to FIG. 4. In the embodiment, a portion of the ultrasound elements 1110 may be utilized for positioning while another portion thereof are used for measurement. For example, a first ultrasound element may first emit a pulse wave for positional alignment of the transducer module 111; and thereafter, a second ultrasound element emit another pulse wave or a continuous wave for blood flow measurement. Likewise, the first ultrasound element may emit a continuous wave for positional alignment of transducer position, followed by the second ultrasound element emit another continuous wave or a pulse wave for blood flow measurement. In another embodiment, each of ultrasound elements 1110 may be switchable between positioning mode and measurement mode. For example, a portion of the ultrasound elements may emit a continuous wave or pulse wave for positional alignment followed by another continuous wave or pulse wave for blood flow measurement, and vice versa.

Arrangement of the ultrasound elements 1110 of the transducer module 11 may vary and be optimized for enhancing accuracy of the alignment and measurement. As exemplified in FIG. 3, the ultrasound elements 1110 may be arranged in a substantially linear pattern, and the distance between two adjacent ultrasound elements 1110 may be optimized for measuring certain characteristics of the target hollow organ (e.g., pulse wave velocity), and may be one to ten centimeters (cm), or preferably 1-2 cm, for common applications, such as measuring the carotid artery or femoral artery. Alternatively, the ultrasound elements 1110 may be arranged orthogonally, as exemplified in FIG. 4C, radially, concentrically, spirally, or in other patterns that allow assessment of the hollow organ from various angles.

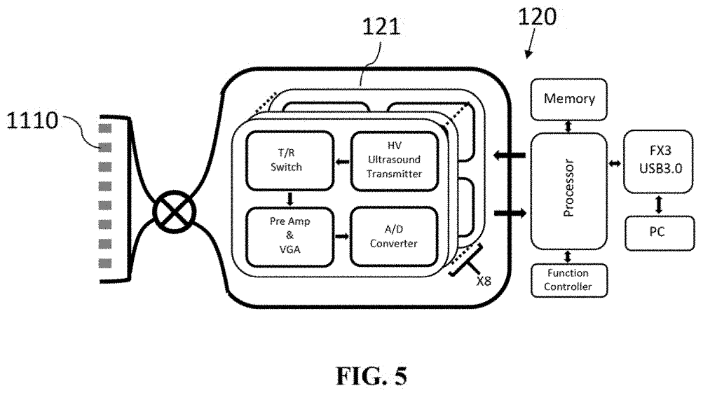

Referring again to FIG. 5. The control device 120 is in communication with the alignment-guidance transducer device 110 and is configured to analyze the ultrasound signals received from the transducer module 111 and transmits instructions to the indicator module 112, therefore guiding the user to align the alignment-guidance transducer device 110 to the extension direction of the target hollow organ 200. The control device 120 may be as simple as a microcontroller, an application specific integrated circuit, a central processor unit, a field programmable gate array (FPGA) or a complex programmable logic device. Alternatively, the control device 120 may be integrated with a mother board or a printed circuit board having a memory or a storage device. Communication between the alignment-guidance transducer device 110 and the control device 120 may be performed via USB, micro USB, serial port, IEEE1394, Bluetooth, Wi-Fi, Infrared, ZigBee, WiMAX, 3G, 40, 4G LTE, 5G, or any other commonly known wired or wireless transmission means. Further, the control device 120 may be coupled with user interfaces or communication modules for further applications. For example, the user interface may be a display to render graphical information of the measured characteristics or a printer to physically deliver a paper report. The communication modules may be coupled with a server or cloud computing device for further data storage or data management. In the embodiment as exemplified in FIG. 5, each of the ultrasound elements 1110 of the transducer module 111 may be in communication with an analog front-end module 121, which includes a high voltage ultrasound transmitter, a T/R switch, a pre-amplifier and variable-gain amplifier (VGA), and an A/D converter. The analog front-end module 121 are in communication with other system hardware, such as processor (e.g., MCU, FPGA), memory (e.g., DRAM), and function controller.

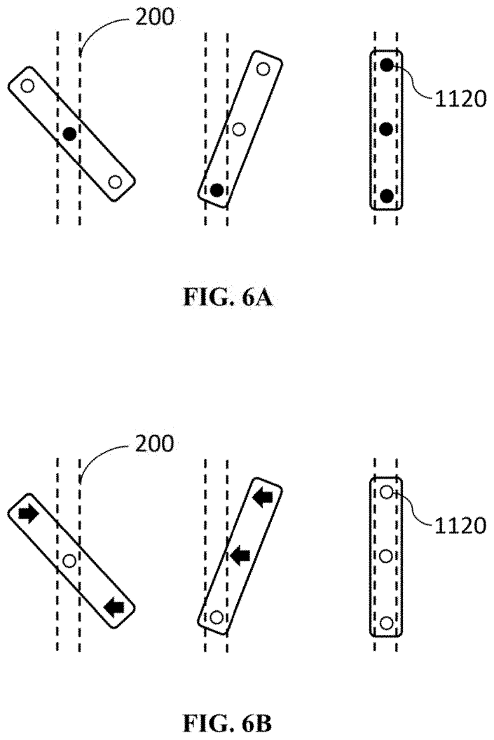

Referring to FIG. 6A and FIG. 6B. The indicator module 112 of the alignment-guidance transducer device 110 includes a plurality of output units 1120 for indicating the state of alignment between the alignment-guidance transducer device 110 and the target hollow organ 200 according to instructions received from the control device 120. The output units 1120 may be visual output units (e.g., light emitting diodes) that generate visible signals and/or audio output units (e.g., speakers) that generates sound signals. The visual output module may be operated in dual mode, discrete scale mode, continuous scale mode, digital mode or directional mode. As exemplified in FIG. 6A, the state of alignment may be indicated by stationary signals, such as solid color lights (e.g., red and green lights) or an on/off signal. As exemplified in FIG. 6B, the alignment state may also be presented by dynamic signals, such as arrow, directional sign, gradient of color, varying light brightness or color intensity, or varying speed of blinking or beeping to indicate suggested direction of movement. In other embodiments, the control device 120 may communicate with an external device, such as a computer, laptop, tablet, or smartphone, as illustrated in FIG. 5, for displaying the state of alignment of the alignment-guidance transducer device 110.

According to an embodiment of the present disclosure, an alignment guidance method of the ultrasound transducer system 100 includes the steps of: (S1) emitting ultrasound waves to a target hollow organ 200 by the ultrasound elements 1110 in the transducer module ill of the alignment-guidance transducer device 110; (S2) receiving ultrasound signals reflected from the target hollow organ 200 by the ultrasound elements 1110; (S3) processing the ultrasound signals by the control device 120 to obtain levels of alignment of the ultrasound elements 1110 to the target hollow organ 200; and (S4) indicating a state of alignment of the ultrasound elements 1110 by the indicator module 112 according to the levels of alignment of the ultrasound elements 1110.

Specifically, prior to starting Step S1 and S2, the alignment-guidance transducer device 110 is held by a user and let stay at a body part of the subject adjacent to the target hollow organ 200. In Step S1, the ultrasound elements 1110 may emit the ultrasound waves individually or in a synchronized or phase-delayed manner. The ultrasound waves emitted by each of the ultrasound elements 1110 may vary, depending on actual implementations; for example, one or more of the ultrasound elements may be configured to emit a continuous wave or pulse wave for positional alignment for a duration, followed by another continuous wave or pulse wave for blood flow measurement for another duration. The ultrasound waves emitted by different ultrasound elements 1110 may vary as well; for example, one of ultrasound element may emit a pulse wave while another emit a continuous wave. Further, when the ultrasound elements 1110 are configured to emit the ultrasound waves individually, the emission may be performed according to a predefined order or sequence.

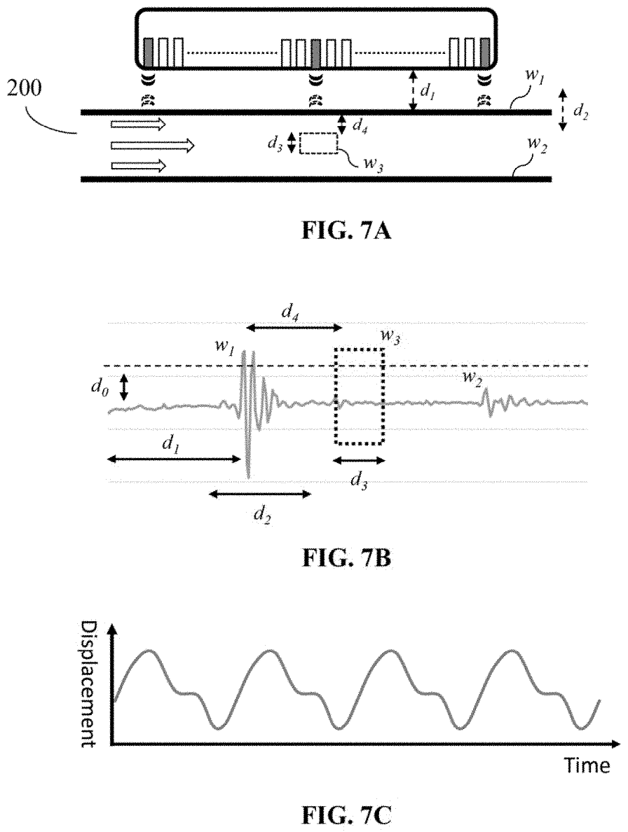

Referring to FIG. 7. In the embodiment, the step S3 includes the steps of: (S31) analyzing the ultrasound signals to obtain one or more characteristics of the target hollow organ 200; and (S32) determining levels of alignment of the ultrasound elements 1110 to the target hollow organ 200 according to the characteristics. Specifically, as exemplified in FIG. 7A, the ultrasound waves emitted by the ultrasound elements 1110 reach various parts of the target hollow organ 200 having fluid flowing therein (direction of the flow is indicated by the hollow arrows). The ultrasound signals reflected from the target hollow organ 200 is exemplified in FIG. 7B. Analysis of the ultrasound signals by the control device 120 reveals various characteristics of the target hollow organ 200, including, for example, the presence of the wall w₁, w₂ of the target hollow organ as determined according to a distance threshold d₀, the distance d₁ between the ultrasound element and the wall w₁, the distance d₂ of displacement of the wall w₂, the size d₃ and depth d₄ of the time window w₃ of the fluid flow in the target hollow organ. As illustrated in FIG. 7C, recording of the ultrasound signals over time further reveals the displacement waveform of the target hollow organ 200.

In Step S3, the levels of alignment of the ultrasound elements to the target hollow organ may be defined by the distances between each of the ultrasound elements and the target hollow organ, the angle formed by the ultrasound elements relative to the extension direction of the target hollow organ, or the signal-to-noise ratio of the received ultrasound signals. Finally, in Step S5, the control device 120 instructs each of the output units of the indicator module to indicate the state of alignment of various parts of the alignment-guidance transducer device 110 according to various levels of alignment of the ultrasound elements by stationary signals and/or to guide the user to move the ultrasound transducer device by dynamic signals. As exemplified in FIG. 6A, each of the output units 1120 is configured to indicate the state of alignment of the ultrasound elements in the corresponding section of the alignment-guidance transducer device 110 to the target hollow organ 200 by two distinct color signals (e.g., green and red signals). Consequently, the user can be informed real-time of the state of alignment of the alignment-guidance transducer device 110 to the extension direction of the target hollow organ 200. Another example as illustrated in FIG. 6B uses dynamic signals to more intuitively inform the user the suggested direction of movement of the alignment-guidance transducer device 110.

In sum, the ultrasound transducer system according to various embodiments of the present invention utilizes an ultrasound transducer device having an intuitive alignment guidance function to facilitate alignment of the transducer device to the target organ and ensure accuracy of pulse wave measurements by the ultrasound transducer system.

Previous descriptions are only embodiments of the present disclosure and are not intended to limit the scope of the present disclosure. Many variations and modifications according to the claims and specification of the disclosure are still within the scope of the claimed disclosure. In addition, each of the embodiments and claims does not have to achieve all the advantages or characteristics disclosed. Moreover, the abstract and the title only serve to facilitate searching patent documents and are not intended in any way to limit the scope of the claimed disclosure.

What is claimed is:

1. An ultrasound transducer device having alignment guidance function, comprising:
a transducer module for emitting ultrasound waves and receiving ultrasound signals reflected from a target hollow organ; and
an indicator module for indicating a state of alignment between the ultrasound transducer device and the target hollow organ, and
wherein the ultrasound transducer device is so configured that the ultrasound transducer device is communicable with a control device capable of processing the ultrasound signals received from the transducer module and transmitting instructions to the indicator module according to the processed ultrasound signals.

2. The ultrasound transducer device according to claim 1, wherein the transducer module comprises a plurality of ultrasound elements for emitting the ultrasound waves and receiving the ultrasound signals.

3. The ultrasound transducer device according to claim 2, wherein each of the ultrasound elements is an actuator element, a sensor element or a transducer element.

4. The ultrasound transducer device according to claim 2, wherein the ultrasound elements emit the ultrasound waves in a synchronized or phase-delayed manner.

5. The ultrasound transducer device according to claim 2, wherein the ultrasound waves emitted by at least a portion of the ultrasound elements are continuous waves or pulse waves.

6. The ultrasound transducer device according to claim 1, wherein the transducer module is configured for detecting one or more characteristics of the target hollow organ.

7. The ultrasound transducer device according to claim 6, wherein the characteristics are at least one of a wall of the target hollow organ and displacement of the wall.

8. The ultrasound transducer device according to claim 1, wherein the transducer module is further configured for measuring one or more characteristics of the target hollow organ.

9. The ultrasound transducer device according to claim 8, wherein the characteristics comprise pulse wave velocity.

10. The ultrasound transducer device according to claim 2, wherein the ultrasound elements are arranged in a substantially linear pattern.

11. The ultrasound transducer device according to claim 2, wherein the ultrasound elements are orthogonally arranged.

12. The ultrasound transducer device according to claim 1, wherein the indicator module comprises a plurality of output units, each of the output units is a visual output unit or an audio output unit.

13. The ultrasound transducer device according to claim 1, wherein the state of alignment is indicated by stationary signals.

14. The ultrasound transducer device according to claim 1, wherein the state of alignment is indicated by dynamic signals.

15. An ultrasound transducer system, comprising:
an alignment-guidance transducer device, comprising:
a transducer module for emitting ultrasound waves and receiving ultrasound signals reflected from a target hollow organ; and
an indicator module for indicating a state of alignment between the alignment-guidance transducer device and the target hollow organ; and
a control device in communication with the alignment-guidance transducer device for processing the ultrasound signals received from the transducer module and transmitting instructions to the indicator module according to the processed ultrasound signals.

16. The ultrasound transducer system according to claim 15, wherein the transducer module comprises a plurality of ultrasound elements for emitting the ultrasound waves and receiving the ultrasound signals.

17. The ultrasound transducer system according to claim 16, wherein each of the ultrasound elements is an actuator element, a sensor element or a transducer element.

18. The ultrasound transducer system according to claim 16, wherein the ultrasound elements emit the ultrasound waves in a synchronized or phase-delayed manner.

19. The ultrasound transducer system according to claim 16, wherein the ultrasound waves emitted by at least a portion of the ultrasound elements are continuous waves or pulse waves.

20. The ultrasound transducer system according to claim 15, wherein the transducer module is configured for detecting one or more characteristics of the target hollow organ.

21. The ultrasound transducer system according to claim 20, wherein the characteristics are at least one of a wall of the target hollow organ and displacement of the wall.

22. The ultrasound transducer system according to claim 15, wherein the transducer module is further configured for measuring one or more characteristics of the target hollow organ.

23. The ultrasound transducer system according to claim 22, wherein the characteristics comprise pulse wave velocity.

24. The ultrasound transducer system according to claim 16, wherein the ultrasound elements are arranged in a substantially linear pattern.

25. The ultrasound transducer system according to claim 16, wherein the ultrasound elements are orthogonally arranged.

26. The ultrasound transducer system according to claim 15, wherein the indicator module comprises a plurality of output units, each of the output units is a visual output unit or an audio output unit.

27. The ultrasound transducer system according to claim 15, wherein the state of alignment is indicated by stationary signals.

28. The ultrasound transducer system according to claim 15, wherein the state of alignment is indicated by dynamic signals.

29. An alignment guidance method implemented by an ultrasound transducer system, the ultrasound transducer system comprising an alignment-guidance transducer device and a control device in communication with the alignment-guidance transducer device, the alignment-guidance transducer device comprising a transducer module and an indicator module, comprising steps of:
(S1) emitting ultrasound waves to a target hollow organ by the transducer module of the alignment-guidance transducer device;
(S2) receiving ultrasound signals reflected from the target hollow organ by the transducer module;
(S3) processing the ultrasound signals by the control device to obtain levels of alignment of the transducer module to the target hollow organ; and
(S4) indicating a state of alignment of the alignment-guidance transducer device according to the levels of alignment of the transducer module by the indicator module of the alignment-guidance transducer device.

30. The alignment guidance method according to claim 29, wherein the step S3 comprises steps of:
(S31) analyzing the ultrasound signals to obtain one or more characteristics of the target hollow organ; and
(S32) determining the levels of alignment of the transducer module to the target hollow organ according to the characteristics.

31. The alignment guidance method according to claim 30, wherein the characteristics comprise wall of the target hollow organ and displacement of the wall.

32. The alignment guidance method according to claim 30, wherein the transducer module comprises a plurality of ultrasound elements for emitting the ultrasound waves and receiving the ultrasound signals, the levels of alignment of the transducer module to the target hollow organ are defined by distances between each of the ultrasound elements and the target hollow organ, the angle formed by the ultrasound elements relative to the extension direction of the target hollow organ, or the signal-to-noise ratio of the received ultrasound signals.

33. The alignment guidance method according to claim 29, wherein the step S3 further comprises a step of:
(S33) processing the ultrasound signals to obtain measurement of one or more characteristics of the target hollow organ.

34. The alignment guidance method according to claim 33, wherein the characteristics comprise pulse wave velocity.
